(19) United States
(12) Patent Application Publication
(10) Pub. No.: US 2020/0411184 A1
(43) Pub. Date: Dec. 31, 2020

(54) MACHINE LEARNING TECHNIQUES FOR UPDATING MEDICAL DECISIONS BASED ON EVENT DATA FROM FIELD DEVICES

(71) Applicant: 3Derm Systems, Inc., Boston, MA (US)

(72) Inventor: Elliot Swart, Boston, MA (US)

(21) Appl. No.: 16/900,812

(22) Filed: Jun. 12, 2020

Related U.S. Application Data
(60) Provisional application No. 62/861,057, filed on Jun. 13, 2019.

(51) Int. Cl. G16H 50/20; G06N 20/00; G16H 10/60

(52) U.S. Cl. CPC G16H 50/20; G16H 10/60; G06N 20/00

(57) ABSTRACT

A medical decision system generates, using a classifier, a medical decision based on an electronic patient record, which is compiled by medical decision system based on existing event data stored in cloud system. The medical decision system establishes a connection with a device at a second time and receives, from the device, additional event data. Each event in the additional event data has a timestamp corresponding to a first time at least a threshold amount of time prior to the second time, and a delta between the first time and the second time occurred at least in part due to the device being incapable of establishing the connection at the first time. The medical decision system determines whether the additional event data is valid. If the additional event data is valid, the medical decision system merges the additional event data into the existing event data and updates the medical decision.

### RELATED APPLICATIONS

This application claims the benefit of Provisional Application No. 62/861,057, filed on Jun. 13, 2019, which is incorporated herein by reference.

### STATEMENT REGARDING FEDERALLY SPONSORED RESEARCH OR DEVELOPMENT

This invention was made with government support under Defense Health Agency Contract #W81XH-19-C-0009 awarded by the Defense Health Agency. The government has certain rights in the invention.

### BACKGROUND

Devices may be used in a variety of scenarios to gather data describing medical conditions of patients. For example, a device may have a certain set of automated diagnosis capabilities that can be performed without connection to a cloud or server. The device may be used by a solider in a field to determine a diagnosis for a fellow soldier that indicates that the fellow solider requires immediate medical attention and needs to be medevacked to a field hospital. However, without connectivity in the field, the data gathered by the device, including time of diagnosis, time of evaluation, and detailed information about the diagnosis, may not be recorded for a detailed record of the patient's medical history.

In another example, devices at rural hospital may also suffer from poor connectivity or low bandwidth, making the devices unable to consistently communicate with other devices or larger hospital systems to share data through a cloud system. If data describing patients from the rural hospital is not added to a larger storage system, patient records may be incomplete from hospital to hospital, resulting in doctors having imperfect data when assessing patients.

### SUMMARY

The disclosure generally relates to the field of cloud systems, and, more particularly, to integrating event data captured by devices, such as field devices storing medical data, into a cloud medical record system. Devices may capture medical data in a plurality of environments, such as within a hospital, a rural clinic, or an isolated field, which results in some devices being disconnected from the cloud medical record system when bandwidth is low or when located remotely. To receive medical data from these devices once connectivity is possible, a medical decision system may verify that the medical data from the devices is valid (i.e., can be trusted as accurate) and merge the medical data with medical data stored on the cloud medical record system such that the cloud medical record system stores the latest medical data for patients. The medical decision system may update medical decisions based on the merged medical data.

More particularly, in some embodiments, the medical decision system generates, using a classifier, a medical decision based on an electronic patient record, which is compiled by medical decision system based on existing event data stored in cloud system. The medical decision system establishes a connection with a device at a second time and receives, from the device, additional event data. Each event in the additional event data has a timestamp corresponding to a first time at least a threshold amount of time prior to the second time, and a delta between the first time and the second time occurred at least in part due to the device being incapable of establishing the connection at the first time. The medical decision system determines whether the additional event data is valid. If the additional event data is valid, the medical decision system merges the additional event data into the existing event data and updates the medical decision based on the merged event data.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF DRAWINGS

FIG. 1 illustrates an example system environment, according to one embodiment.

FIG. 2 is a high-level block diagram illustrating a detailed view of a medical decision system, according to one embodiment.

FIG. 3A illustrates the interactions that take place between different entities of FIG. 1 for sending decision event data for display, according to one embodiment.

FIG. 3B illustrates the interactions that take place between different entities of FIG. 1 for updating a medical decision, according to one embodiment.

FIG. 4 is a high-level block diagram illustrating physical components of a computer used as part or all of the medical decision system from FIG. 1, according to one embodiment.

FIG. 5 is a flowchart illustrating a process of updating a medical decision, according to one embodiment.

The figures depict embodiments of the present invention for purposes of illustration only. One skilled in the art will readily recognize from the following description that alternative embodiments of the structures and methods illustrated herein may be employed without departing from the principles of the invention described herein.

### DETAILED DESCRIPTION

### System Overview

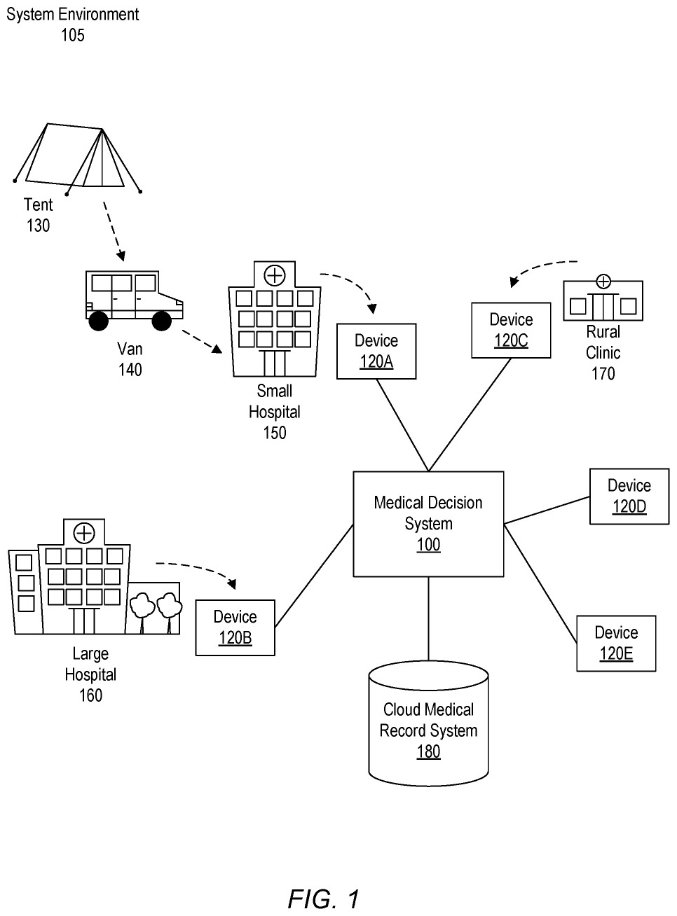

FIG. 1 illustrates an example system environment 105, according to one embodiment. The medical device system 100 stores and synchronizes data across deployments (i.e., application running on devices 120) and enforces security checks. The medical device system 100 may include components such as a cloud medical record system 180 and a plurality of devices 120. In some embodiments, these components may be connected through a standard internet connection in which each component has direct access to cloud server. In other embodiments, the components may operate in an offline environment such that each component is self-sufficient and synchronize captured data to when connection is available. In further embodiments, the components may operate in a mesh network environment and communicate within one another to compile data. These various components are now described in additional detail.

The devices 120 are computing devices such as smart phones, laptop computers, desktop computers, or any other device 120 that is a physical or logical entity that can support a deployment for the medical decision system 100 and record and transmit data. For example, a device 120 may be a skin imaging device for remote skin imaging at a remote location, such as a battlefield, on a mountain, or in a desert. In another example, a device 120 may be a local server that provides caching and compute acceleration to a healthcare facility. A deployment is defined as a particular application (or integrated set of applications) running on a single device 120A. Each deployment describes an implementation of one or more of the components in FIG. 1, and every deployment is associated with a trusted signing key for a user, a device 120A, or both. A signing key may be associated with a validity period of when the signing key may be used for a device 120A or user. For instance, data captured on a device 120A may only be valid during the validity period, and if the data is generated outside of the validity period, then medical record system 100 will not trust the data for use in electronic patient records and medical decisions. In some embodiments, the validity period is an amount of time device 120A can go without connecting to medical decisions system 100 for medical decision system 100 to still trust data generated by device 120A. The connecting logic between components in the system environment 100 is consistent regardless of deployment. Examples of deployments include the cloud medical record system or a mobile application.

Devices 120 records event data describing events, which are timestamped records of a patient's medical conditions. The term event, as used herein, may refer to pieces of data recorded at particular times. Each event includes a unique identifier and a timestamp, both of which are assigned at time of creation of the event and are never changed. For example, a first event may indicate that a patient was diagnosed with a sinus infection at 6:03 pm, and a second event may indicate that the patient was prescribed medicine for the sinus infection at 6:07 pm on the same day. An event also includes a token provided by a user of a device 120A and a request signature signed by the device 120A and/or the user to indicate that the user trusted and signed the event at the device 120A. Further, each event may also be considered a request for the event to be stored in a trusted event store based on the requesting signature.

Devices 120 each maintain a subset of medical decision system 100 running locally. However, the devices 120 may be directly connected to the medical decision system 100 at some times but not others. For instance, device 120A may lack the capability to establish a connection with the medical decision system 100 at all times. Devices 120 may be temporarily disconnected to medical decision system 100, may be connected to medical decision system 100 via a local network that experiences frequent outages, or may only be able to transmit data manually (i.e., through a USB). Devices 120 each synchronize their subset of medical decision system 100 when connectivity to the full medical decision system 100 or another device 120 with a subset of medical decision system 100 is available. For example, device 120A may initially be used in a tent 130 in the wilderness to record and locally store event data describing a patient's medical condition using a local subset of medical decision system 100. While in the tent 130, device 120A may have low bandwidth due to its remote location and be unable to connect to an internet server to send data to and synchronize with medical decision system 100. After transferring device 120A in van 140 to small hospital 150, device 120A may be connected to medical device system 100 manually (e.g., a USB connection) or via a network to transmit the event data.

In another example, large hospital 160 may use device 120B to record event data about a plurality of patients who visit large hospital 160. The device 120B may have an established connection with medical decision system 100 such that device 120B may send event data to medical decision system 100 in real-time as it is recorded. A patient who is a regular at the large hospital may have an electronic record stored in cloud medical record system 180, which medical decision system 100 may access upon request of device 120B when the patient visits large hospital 160. However, when the patient goes to rural clinic 170 while on a camping trip, the rural clinic may need to establish a connection between device 120C and medical decision system 100 to retrieve the patient's electronic patient record on medical decision system 100 and record new event data for the patient. Alternatively, if device 120C could connected with another device 120D that has recently synchronized with medical decision system 100 to retrieve the patient's electronic patient record.

The medical decision system 100 accesses and stores electronic patient records in cloud medical record system 180. In some embodiments, cloud medical record system 180 is a separate entity from the medical decision system 100, and in other embodiments, cloud medical record system 180 is incorporated into medical decision system 100. An electronic patient record is a log of event data pertaining to a patient received from one or more devices 120, and an electronic patient record may comprise multiple fields associated with particular types of event data in JSON format. Event data includes events, which are pieces of data that are generated by devices 120 and transmitted to other devices 120. Each event is associated with a timestamp and an identifier and may refer to multiple fields, such that multiple events may reference the same field. The timestamp for an event is used as the fundamental source of truth for when the event occurred.

To ensure the validity of the timestamp, the timestamp must be generated from a trusted device 120 or verified at when being synched by medical decision system 100. Either an intentional or unintentional error in the clock of the device 120 could cause strange behavior. In some embodiments, the timestamp may be associated with an additional field, such as the last Network Time Protocol (NTP) sync or sync information. This expands the size of the event and is used only when potential errors are expected. However, it still relies on the trustworthiness of device 120 that issued the timestamp. The risk of intentional tamper is largely mitigated by user/device logging, such that any tampering can be easily identified. For devices 120 where the clock is not definitively trusted, medical decision system 100 sets the timestamp or verify it is within proper bounds.

In some embodiments, medical decision system 100 stores events in an event store within cloud medical record system 180 and indexed by identifier. In further embodiments, the events may be indexed by other indices that are highly dependent on how the event store will be used. The event store is idempotent, and medical decision system 100 does not add events with duplicate identifiers to the cloud medical record system and can handle duplicates without changing behavior.

Event data for a plurality of patients may be received by medical device system 100, materialized into a single electronic patient record for each patient, and stored in cloud medical record system 180. A materialized view of the electronic patient record is updated in a highly systematic way when new data comes in. Integrating events into materialized views follows ACID 2.0 in order to allow events to be added to the materialized view in any order. This may be done using the current state of the record and just a single event, in a reduce operation. This may not always be possible for complex queries. In addition, multiple materialized views may be optimized for different purposes, which can be critical when one device 120 requires detailed audit information and another requires only the latest state of an application on device 120. In some embodiments, the medical decision system 100 loads event data into a relational database for use in arbitrary queries, which is also a valid materialization of an event log, but it does not rely on the event data in this relational database as the “true” state of the electronic patient records.

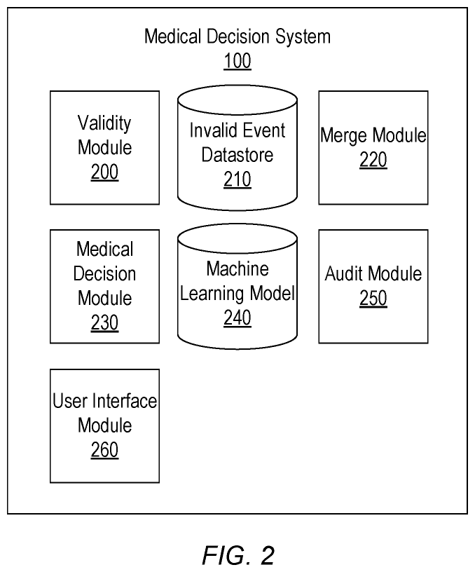

FIG. 2 is a high-level block diagram illustrating a detailed view of the medical decision system 100, according to one embodiment. Medical decision system may be located on a physical device 120 accessible by one or more other devices 120. Medical decision system 100 includes validity module 200, invalid event datastore 210, merge module 220, medical decision module 230, machine learning model 240, audit module 250, and user interface module 260. These various components are now described in additional detail.

Validity module 200 determines the validity of events within event data received from one or more devices 120 and in some embodiments, validity module 200 also determines whether the event data includes duplicate events already received at medical decision system 100 since duplicate events waste network bandwidth and processing time. In one embodiment, validity module 100 deduplicates by first receiving, from device 120A, a list of identifiers of events in the event data over a list and sends to the device a list of identifiers of events already present in event data stored in cloud medical record system 180. Device 120A may then only send events with identifiers that present in the stored event data. In another embodiment, validity module 120 may determine a last time device 120A synched its event data with medical decision system 100 and only receive, from device 120A, event data taken after the last time. In some embodiments, validity module 200 may store a synchronization record in an optional event store, which may be part of validity module 200 or cloud medical record system 180. The synchronization record may be recorded as a directed graph.

Validity module 200 determines the validity of events within event data received from one or more devices 120 to ensure the security of event data when transferred to medical decision system 100. Validity module 200 receives event data from a device 120 along with embedded tokens. A token is a gold standard method for decentralized authorization where a trusted party signs a set of data and another party verifies the signature of the trusted party before trusting that data for authorization. JSON Web Tokens (JWT) are an optimal way to do this, as JSON Web Tokens have very small overhead compared to other technologies, such as SAML. JSON Web Tokens may be nested, such that one token contains multiple inner tokens. Device 120A may be associated with one or more signing keys for a user and/or a device that also includes an expiration timestamp. The other tokens received by validity module from device 120A will be tied to the validity of device identifier token.

Validity module 200 determines whether the event data is valid based on an attestation, which is a verification that event data can be used as the “ground truth.” which is made using a signing key from the device 120A that verifies permissions for the event data. A user of device 120A may sign the token with the event data with the signing key, indicating who made the attestation. The event data may be signed, by device 120A, by converting the event data into a canonical JSON format, obtaining a hash of the canonical request, integrating a device identifier related to device 120A into the hash, and digitally signing the hash with the signing key of device 120A. The event, including the signed hash, can then be submitted to medical decision system 100. Validity module 200 may be located on a device 120A, and validity module 200 verifies event data is valid based on the latest available data about the identity and public signing keys of all or a subset of devices, Cloud medical record system 180 contains a master record, both of event data and all devices (with their public keys), which will be synchronized to device 120A. Validity module 200 confirms that the signature in the token matches with the signing key for the device in the attestation, and if so, the event data may be considered valid. For example, if the signing key for device 120A is used to generate the token for event data has an expiration timestamp earlier in time than the timestamp of the event data, then validity module 100 would consider the events in the event data invalid.

Validity module 200 may store valid events in cloud medical record system 180 or at a local datastore, known as a trusted event store in some embodiments. Further, validity module 200 stores invalid events from the event data in invalid event store 210. Invalid events are events that validity module 300 could not authenticated. The invalid events in invalid event store 210 may be synced with other invalid events stored in cloud medical record system 180. Validity module 200 may remove invalid events from the invalid event datastore 210 after manual validation is entered and received from user interface module 260.

Merge module 220 receives valid events and merges the valid events with event data in a patient's electronic record retrieved from cloud medical record system 180. For each valid event, merge module 220 trusts the timestamp of the valid event given that validity module 200 already verified the event and assumes that the timestamp corresponds to a time space of the event data stored in cloud medical record system 180. In some embodiments, if the timestamp was manually set at the device 120A, merge module 220 synchronizes the timestamp with a reference clock at medical decision system 100.

In some embodiments, merge module 220 may merge the valid events into the electronic patient record in any order. Merge module 220 receives event data in a patient medical record from cloud medical record system 180 and determines whether valid events associated with the patient are associated with a field of the patient medical record. Merge module 220 retrieves an event currently in the field from the patient medical record. If a timestamp of the event currently in the field is earlier than the timestamp of the valid event, the merge module replaces the event currently in the field with the valid event. Otherwise, merge module 220 leaves the event currently in the field in the electronic patient record. In some embodiments, merge module 220 may still keep a record that the valid event occurred in cloud medical record system 180. In particular, merge module 220 may preserve all events pertaining to a field even if the event data seems to contradict the event previously in the field, such that medical decision system 100 could still retrieve both events and information describing what device 120 assigned each event.

Further, if a valid event is not associated with a field, merge module 220 may create a field for the valid event in the electronic patient record or may merely merge the valid event into the patient electronic record based on the timestamp. Merge module 220 stores the electronic patient record with the merged event data in cloud medical record system 180 along with a merge timestamp indicating when the event data was merged. In some embodiments, merge module 220 may send an indication to medical decision module 230 that a patient's electronic patient record contains newly merged event data and requires an updated medical decision.

Medical decision module 230 generates medical decisions for a patient based on the patient's electronic patient record. Medical decision module 230 may be located within medical decision system 100, or, in some embodiments, may be located onboard device 120B, which is a LAN-connected device 120B in this embodiment. If located on device 120B, medical decision module 230 may request the electronic patient record from medical decision system 100 and generate a medical decision from device 120B. Otherwise, medical decision module 230 may generate the medical decision and send it to device 120B. Further, in some embodiments, the request may be received from the same device 120B that sent the event data, or may be a different a different device 120B at another physical location. Medical decision module 230 may request a specific subset of event data in the electronic patient record relevant to the medical decision being requested. For example, if a medical decision about a patient's eyesight is requested, medical decision module 230 may only retrieve event data pertaining to the patient's eyesight rather than event data about the patient's dental health.

Medical decision module 230 receives a request from user interface module 260, as entered from device 120B, for a medical decision for a patient based on the patient's electronic patient record. In some embodiments, medical decision module 230 generates a medical decision in response to receiving an indication from merge module 220 that a patient's electronic patient record contains newly merged evet data. Medical decision module retrieves an electronic patient record from cloud medical record system 180 and inputs event data from the electronic patient record to machine learning model 240. Machine learning model 240 may be one or more machine-learned classifiers trained to generate a medical decision based on event data in electronic patient records. Machine learning model 240 may be trained on training data including a set of diagnoses labelled with event data from electronic patient records. The training data may be manually or automatically labelled.

Medical decision module 230 receives outputs from machine learning model 240 indicating percentage matches to one or more diagnoses. In some embodiments, machine learning model 240 may directly output one or more diagnoses. Medical decision module 230 selects one or more of the diagnoses for the patient based on the percentage matches. For example, in one embodiment, medical decision module selects the diagnosis with the highest percentage match. In another embodiment, medical decision system 100 selects all diagnoses above a threshold percentage match as diagnoses. In an embodiment, where multiple diagnoses are above the threshold percentage match, medical decision system 100 indicates that a diagnosis cannot be made (e.g., where two mutually exclusive diagnoses are identified). Medical decision module 230 sends the diagnoses to user interface module 260 as the medical decision for the patient.

Medical decision module 240 stores the one or more diagnoses as medical decisions in cloud medical record system 180 timestamped with a decision timestamp based on when medical decision system 240 selected the diagnosis. For example, an electronic patient record may contain, as event data, a multitude of images of a patient's eyes taken by an optometrist, as well as a description of the patient's vision taken the same day by the optometrist. Medical decision module 230 inputs the event data to machine learning model 240 and receives a percentage match to the diagnosis “astigmatism,” which is recorded at a medical decision for the patient in cloud medical record system 180. This recording may be considered an event on which further medical decisions may be based.

Audit module 250 receives audit indications from one or more devices 120 connected to medical decision system 100 via user interface module 260. An audit indication indicates a medical decision in an electronic patient record that a user of device 120B wants to audit. Audit module 250 retrieves, from cloud medical record system 180, a decision timestamp associated with the medical decision. Audit module 250 retrieves merge timestamps for the electronic patient record and determines a comparison merge timestamp indicating when event data was last merged into the patient electronic record before the medical decision was generated. In an alternate embodiment, audit module 250 may also retrieve identifiers for events used to generate the medical decision, which may be stored alongside the medical decision to enable auditability of the medical decision. Audit module 250 retrieves decision event data corresponding to the merge timestamp (or, in some embodiments, the identifiers), which is the event data in the electronic patient record that was used to make the medical decision. Audit module 250 may send the decision event data to user interface module 260 for display on device 120B such that a user may trace back through the event data to determine how the medical decision was made and if it was a fair medical decision to make based on the event data.

In some embodiments, audit module 250 may determine whether the medical decision was correct given decision event data. Audit module 250 may send the decision event data to medical decision module 240 to generate a new medical decision to verify against the medical decision. If the medical decision was correct, audit module 250 may send an indication to user interface module 260 indicating that the medical decisions was correct, and if not, audit module 250 may send an indication to user interface module 260 indicating that the medical decisions was incorrect.

For example, a medical decision that a patient does not have astigmatism may have been determined based on event data that included 7 images of the patient's eyes. However, the patient's current electronic patient record may include 8 images, the newest of which shows that the patient has astigmatism. Audit module 250 may determine that the medical decision was made correctly since the newest image showing the astigmatism was not available in cloud medical record system 180 when the medical decision was made. In another example, the medical decision may be have generated at device 120B while device 120B was disconnected from medical decision system 100. Though cloud medical record system 180 may have had the 8 images when the medical decision was generated, if device 120B only had the 7 images, audit module 250 may determine that the medical decision was still made correctly given the event data available at device 120B when the medical decision was made.

Audit module 250 may verify medical decisions made on disconnected devices. In particular, if a medical decision was made while device 120B was disconnected from medical decisions system 100, audit module 250 retrieves the most up-to-date event data from cloud medical record system 180 and sends the event data to medical decision module 240. If the new medical decision generated by medical decision module 240 is different from the original medical decision, audit module 250 may send an indication to user interface module 260 to display on devices 120 associated with a doctor and/or the patient that the medical decision is incorrect.

User interface module 260 creates user interfaces to send devices 120 for display. User interface module 260 may create interactive user interfaces to receive or display electronic patient records, medical decisions, indications, alerts, requests, or other event data. In some embodiments, user interface module 260 may receive invalid events from invalid event store 210 to send to device 120C for manual verification of the invalid events via an interactive display of device 120C. In other embodiments, user interface module 260 may request and receive medical decisions for display to a user of device 120C. In further embodiments, responsive to receiving an audit indication from device 120D, user interface module 260 may receive indications that a medical decision was correct or incorrect, which user interface module 260 may incorporate into a user interface to be sent to device 120D.

### Example Interactions

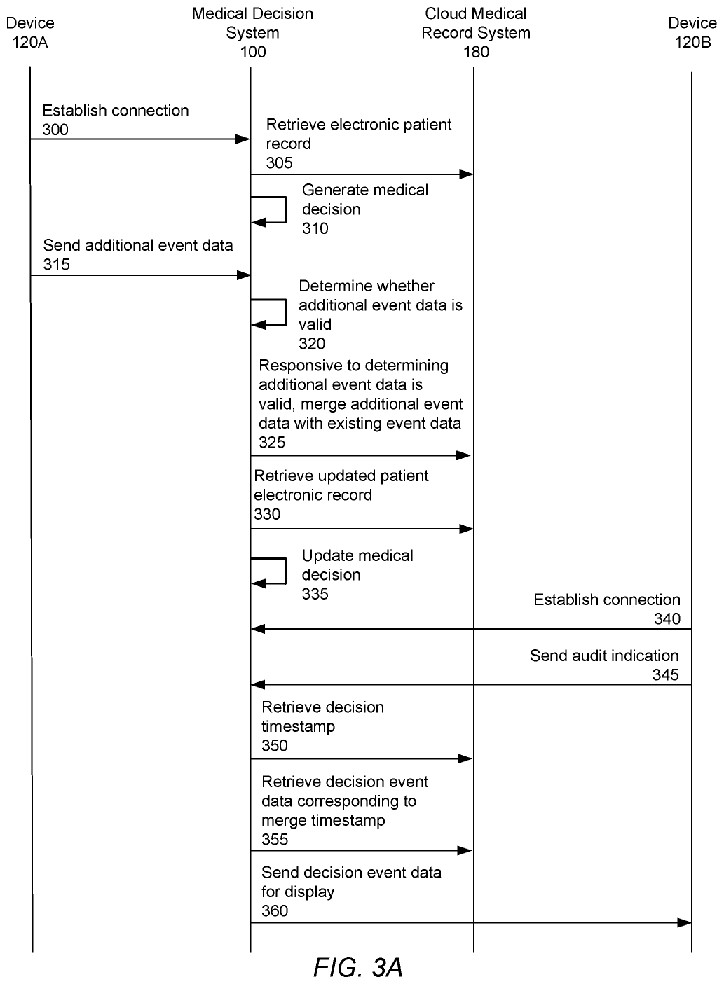

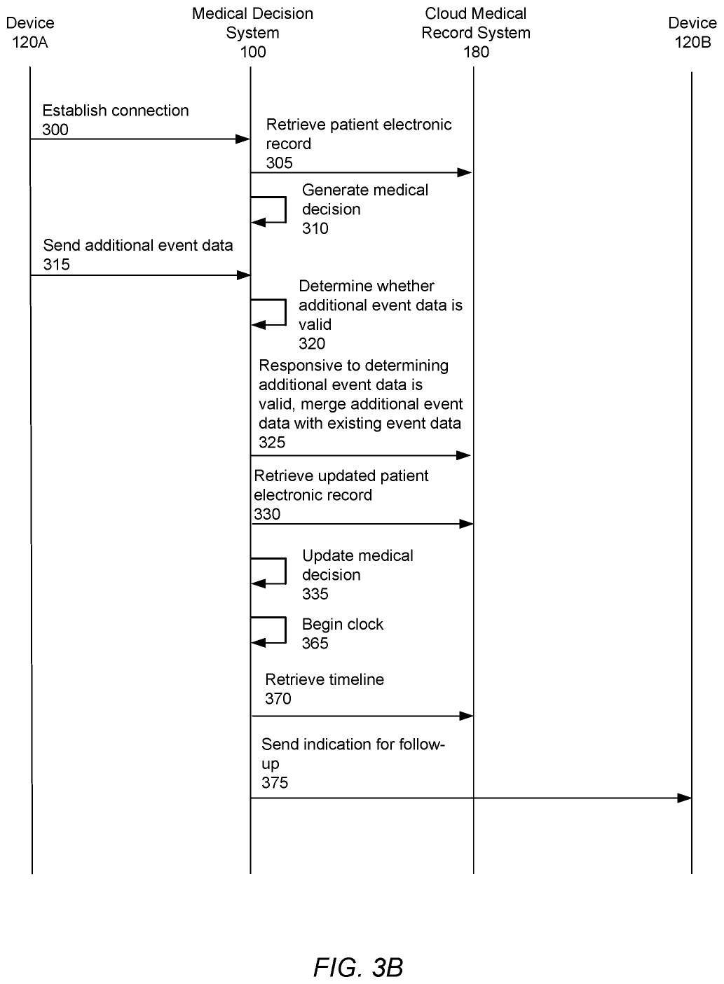

FIG. 3 illustrates the interactions that take place between different entities of FIG. 1, according to one embodiment. The entities in FIG. 3 include device 120A, medical decision system 100, cloud medical record system 180, and device 120B. It is appreciated that although FIG. 3 illustrates a number of interactions according to one embodiment, the precise interactions and/or order of interactions may vary in different embodiments. For example, in some embodiments, FIG. 3 may include more devices 120.

In FIG. 3A, device 120A establishes 300 a connection with the medical decision system 100. Device 120A may not have been previously connected to medical decision system 100 due to being previously incapable of establishing a connection or may have an intermittent connection with medical decision system 100 due to low bandwidth. For example, device 120A may record event data at a first time before establishing a connection at a second time, where the delta between the first and second time is due to the inability of device 120A to connect to medical decision system 100 at the first time.

Medical decision system 100 retrieves 305 a patient electronic record associated with a patient from the cloud medical record system 180. Based on this electronic patient record, the medical decision system 100 generates 310 a medical decision for the patient using machine learning model 240. For example, if the patient's electronic patient record indicates that the patient has a rash, medical decision system 100 may generate a medical decision that a patient has contact dermatitis based on the electronic patient record. Device 120A sends 315 additional event data associated with the patient to medical decision system 100, and medical decision system 100 determines whether the additional event data is valid using validity module 200.

Responsive to determining that the additional event data is valid, medical decision system 100 merges the additional event data with existing event data for the patient in the electronic patient record stored in cloud medical record system 180. Medical decision system 100 retrieves the updated electronic patient record from cloud medical record system 180 and updates the medical decision based on the updated electronic patient record. Returning to the previous example, if the information about the rash was changed based on the merged data, medical decision system 100 may determine that the patient does not have contact dermatitis and actually has heat rash.

The device 120B establishes 340 a connection with medical decision system 100. Device 120B may have been previously connected with medical decision system 100 or may be newly connected to medical decision system 100 to send 345 an audit indication. Device 120B sends 345 the audit indication that requests event data regarding the medical decision for auditing purposes. Medical decision system 100 retrieves 350, from cloud medical record system 180, a decision timestamp associated with the medical decision indicating when the medical decision was made. Medical decision system 100 also retrieves 355 decision event data corresponding to a merge timestamp. The decision event data includes events used to generate the medical decision that were available at the time of the merge timestamp. Medical decision system 100 sends 360 the decision event data for display at device 120B, which may be used for auditing the medical decision.

In some embodiments, medical decision system 100 may determine that the medical decision was correct based on the decision event data available when the medical decision was made and may send, for display with the decision event data, an indication that the medical decision was correct to device 120B. In other embodiments, medical decision system 100 may determine that the medical decision was incorrect based on the decision event data available when the medical decision was made. The medical decision system 100 sends, for display with the decision event data, an indication that the medical decision was incorrect to device 120B given the event data available at the time the medical decision was made.

In FIG. 3B, interactions 300-335, between device 120A, medical decision system 100, and cloud medical record system 180, have the same weight of description as described above with respect to FIG. 3A, thus resulting in an updated medical decision based on the updated patient electronic record. Upon updating the medical decision, medical decision system 100 begins 365 a clock associated with the patient. The clock indicates when the medical decision was made. In some embodiments, if the medical decision was not changed even after the patient medical record was updated, medical decision system 100 may adjust the clock to correspond to the timestamp of the medical decision.

Medical decision system 100 retrieves 370 a timeline associated with the medical system. For example, a medical decision that indicates the patient has vision issues may be associated with a timeline that includes a follow-up appointment for the patient with an optometrist within a 3-month timeframe to ensure that the vision issues are addressed. Medical decision system 100 sends 375 an indication to device 120B, which is associated with the patient in FIG. 3B, recommending a follow-up appointment with an optometrist within 3 months. In some embodiments, medical decision system 100 may also alert an optometrist associated with the patient indicating that the patient has vision issues that need to be addressed.

### Computer Diagram

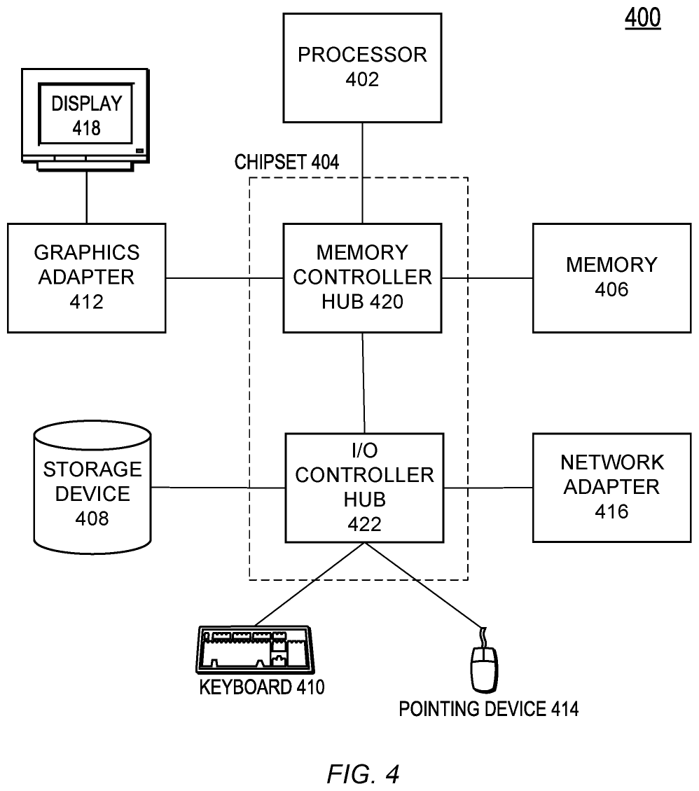

FIG. 4 is a high-level block diagram illustrating physical components of a computer used as part or all of the medical decision system from FIG. 1, according to one embodiment. Illustrated are at least one processor 402 coupled to a chipset 404. Also coupled to the chipset 404 are a memory 406, a storage device 408, a graphics adapter 412, and a network adapter 416. A display 418 is coupled to the graphics adapter 412. In one embodiment, the functionality of the chipset 404 is provided by a memory controller hub 420 and an I/O controller hub 422. In another embodiment, the memory 406 is coupled directly to the processor 402 instead of the chipset 404.

The storage device 408 is any non-transitory computer-readable storage medium, such as a hard drive, compact disk read-only memory (CD-ROM), DVD, or a solid-state memory device. The memory 406 holds instructions and data used by the processor 402. The graphics adapter 412 displays images and other information on the display 418. The network adapter 416 couples the computer 400 to a local or wide area network.

As is known in the art, a computer 400 can have different and/or other components than those shown in FIG. 4. In addition, the computer 400 can lack certain illustrated components. In one embodiment, a computer 400 acting as a server may lack a graphics adapter 412, and/or display 418, as well as a keyboard or pointing device. Moreover, the storage device 408 can be local and/or remote from the computer 400 (such as embodied within a storage area network (SAN)).

As is known in the art, the computer 400 is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device 408, loaded into the memory 406, and executed by the processor 402.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

### Medical Decision Process

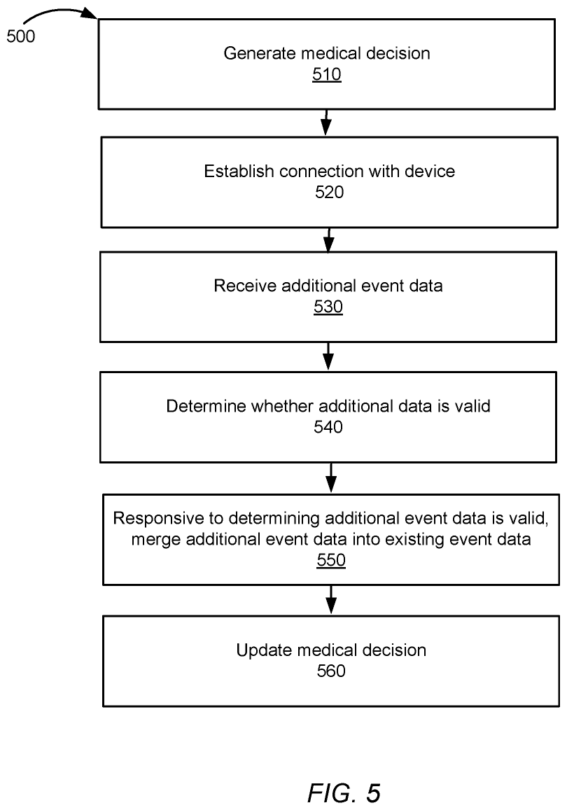

FIG. 5 is a flowchart illustrating a process 500 of updating a medical decision, according to one embodiment. It is appreciated that although FIG. 5 illustrates a number of steps according to one embodiment, the precise steps and/or order of steps may vary in different embodiments. For example, in some embodiments, the first step of the process 500 may be to retrieve an electronic medical record from cloud medical record system 180. Various modules may be used to perform elements of process 500, and may be executed by one or more processors 402.

As depicted in the process 500 of FIG. 5, medical decision system 100 generates 510 a medical decision based on an electronic patient record by executing one or more classifiers, such as machine learning model 240. The medical decision may be generated using medical decision module 230. In some embodiments, the electronic patient record is compiled by merge module 220 based on existing event data stored in cloud medical record system 180. The classifier may be trained to generate medical decisions based on event data. Medical decision system 100 establishes 520 a connection with device 120A at a second time and receives 530, from device 120A, additional event data. Each event in the additional event data has a timestamp corresponding to a first time at least a threshold amount of time prior to the second time, where the delta between the first time and the second time occurred at least in part due to device 120A being incapable of establishing the connection at the first time.

Medical decision system 100 determines 540 whether the additional event data is valid via validity module 200, and, responsive to determining that the additional event data is valid, medical decision system 100 merges 550, using merge module 220, the additional event data into the existing event data in cloud medical record system 180. Medical decision system 100 updates 560 the medical decision based on the merged event data via medical decision module 230. In some embodiments, medical decision system 100 may transmit, via user interface module 260, the updated medical decision to a display 418 of the device 120A, which may store the updated medical decision in its memory 406. In other embodiments, medical decision system 100 may store the merged event data in cloud medical record system 180, which is accessible by a plurality of other devices 120.

In some embodiments, the existing event data corresponds to a field in the electronic patient record. To merge 550 the additional event data into the existing event data via merge module 220, medical decision system 100 receives a corresponding timestamp of corresponding event data in the field of the electronic patient record. Medical decision system 100 compares the timestamp of the additional event data to the corresponding timestamp. Responsive to determining that the timestamp is later than the corresponding timestamp, medical decision system 100 replaces the corresponding event data with the additional event data in the field of the electronic patient record.

In some embodiments, medical device system 100 may receive 530 the additional event data from device 120A by receiving, from device 120A, one or more events in the additional event data, where each event has a timestamp manually set at device 120A. Medical decision system 100 synchronizes the timestamps with a reference clock of a device 120B on which the medical decision was made. This allows the additional event data, when merged by merge module 220, to have timestamps consistent with other event data used by medical decision system 100 and stored at cloud medical record system 180. In other embodiments, medical device system 100 may receive 530 the additional event data from device 120A by receiving one or more identifiers associated with events in the additional event data and determine a new set of identifiers each corresponding to events not sent from device 120A to be merged with the existing event data. Medical decision system 100 requests responsive events from device 120A corresponding to the new identifiers in the set and receives, from device 120A, the responsive events corresponding to the new identifiers.

In some embodiments, to determine the validity of the additional event data via validity module 200, medical decision system 100 receives, for each event in the additional event data, an attestation associated with a signing key and an issue timestamp. Medical decision system 100 determines, for each of the one or more received attestations, whether the attestation is valid based on its issue timestamp, its signing key, and a validity period of a signing key for device 120A. In some embodiments, each device 120 includes its own attestation service, such that devices 120 can relay and verify event data between one another before the event data is sent to medical decisions system 100. If an attestation is invalid, medical decision system 100 stores the event associated with the attestation in invalid event datastore 210 to be manually validated. If the attestation is valid, medical decision system 100 stores the event associated with the attestation in a local event store (e.g., memory 406) of a device 120B which made the medical decision.

### Summary

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method comprising:
generating, using a classifier, a medical decision based on an electronic patient record, the electronic patient record compiled based on existing event data stored at an event database;
establishing a connection with a device at a second time;
receiving, from the device, additional event data, each event in the additional event data having a timestamp that corresponds to a first time at least a threshold amount of time prior to the second time, the delta between the first time and the second time having occurred at least in part based on the device having been incapable of establishing the connection;
determining whether the additional event data is valid;
responsive to determining that the additional event data is valid, merging the additional event data into the existing event data at the event database; and
updating the medical decision based on the merged event data.

2. The method of claim 1, further comprising:
receiving an audit indication for the medical decision;
retrieving a decision timestamp associated with the medical decision;
retrieving, from the electronic patient record, decision event data corresponding to a merge timestamp, the merge timestamp indicating when the decision event data was available for determining medical decisions; and
sending the decision event data for display on the medical audit device.

3. The method of claim 2, further comprising:
determining that the medical decision was correct based on the decision event data available when the medical decision was made; and
sending, for display with the decision event data, an indication that the medical decision was correct given the event data available when the medical decision was made.

4. The method of claim 2, further comprising:
determining that the medical decision was incorrect based on the decision event data available when the medical decision was made; and
sending, for display with the decision event data, an indication that the medical decision was incorrect given the event data available when the medical decision was made.

5. The method of claim 1, wherein the existing event data corresponds to a field in the electronic patient record and merging the additional event data into the existing event data based on the timestamp comprises:
receiving a corresponding timestamp of corresponding event data that includes event data that refers to the field;
comparing the timestamp of the additional event data to the corresponding timestamp of the corresponding event data that includes event data that refers to the field; and
responsive to determining that the timestamp is later than the corresponding timestamp, replacing the corresponding event data with the additional event data in the field of the electronic patient record.

6. The method of claim 1, wherein receiving, from the device previously incapable of providing event data, additional event data comprises:
receiving, from the device, one or more events in the additional event data, each event having a timestamp manually set at the device; and
synchronizing the timestamps with a reference clock of a device with which the medical decision was made.

7. The method of claim 1, wherein determining the validity of the additional event data comprises:
receiving, for each event in the additional event data, an attestation associated with a signing key and an issue timestamp;
determining, for each of the one or more attestations, whether the attestation is valid based on its issue timestamp, its signing key, and a validity period of the signing key associated with the device.

8. The method of claim 7, further comprising:
responsive to determining that a attestation is invalid, storing the event associated with the attestation in an invalid event store, wherein events in the invalid event store may be manually validated.

9. The method of claim 7, further comprising:
responsive to determining that a attestation is valid, storing the event associated with the attestation in a local event store of a device with which the medical decision was made.

10. The method of claim 1, further comprising:
storing the merged event data in a cloud medical record system, the cloud medical record system accessible by a plurality of connected devices and intermittently accessible to plurality of previously disconnected devices.

11. The method of claim 1, wherein receiving, from a device previously incapable of providing event data, additional event data comprises:
receiving one or more event identifiers associated with events in the additional event data;
determining a set of new event identifiers, the new event identifiers corresponding to events not sent from the device to be merged with the existing event data;
requesting responsive events from the device corresponding to new event identifiers in the set of new event identifiers; and
receiving, from the device, the responsive events corresponding to the new event identifiers in the set of new event identifiers.

12. The method of claim 1, wherein the classifier is a machine learning model trained to generate medical decisions based on event data.

13. A computer program product for updating a medical decision, the computer program product comprising a computer-readable storage medium containing computer program code for:
generating, using a classifier, the medical decision based on an electronic patient record, the electronic patient record compiled based on existing event data stored at an event database;
establishing a connection with a device at a second time;
receiving, from the device, additional event data, each event in the additional event data having a timestamp that corresponds to a first time at least a threshold amount of time prior to the second time, the delta between the first time and the second time having occurred at least in part based on the device having been incapable of establishing the connection;
determining whether the additional event data is valid;
responsive to determining that the additional event data is valid, merging the additional event data into the existing event data at the event database; and
updating the medical decision based on the merged event data.

14. The computer program product of claim 13, wherein the computer-readable storage medium further contains computer program code for:
receiving an audit indication for the medical decision;
retrieving a decision timestamp associated with the medical decision;
retrieving, from the electronic patient record, decision event data corresponding to a merge timestamp, the merge timestamp indicating when the decision event data was available for determining medical decisions; and
sending the decision event data for display on the medical audit device.

15. The computer program product of claim 13, wherein the existing event data corresponds to a field in the electronic patient record and wherein merging the additional event data into the existing event data based on the timestamp further comprises:
receiving a corresponding timestamp of corresponding event data that includes event data that refers to the field;
comparing the timestamp of the additional event data to the corresponding timestamp of the corresponding event data that includes event data that refers to the field; and
responsive to determining that the timestamp is later than the corresponding timestamp, replacing the corresponding event data with the additional event data in the field of the electronic patient record.

16. The computer program product of claim 13, wherein receiving, from the device previously incapable of providing event data, additional event data further comprises:
receiving, from the device, one or more events in the additional event data, each event having a timestamp manually set at the device; and
synchronizing the timestamps with a reference clock of a device with which the medical decision was made.

17. A computer system comprising:
one or more computer processors; and
a non-transitory computer-readable storage medium storing instructions that when executed by the one or more computer processors, performs actions comprising:
generating, using a classifier, a medical decision based on an electronic patient record, the electronic patient record compiled based on existing event data stored at an event database;
establishing a connection with a device at a second time;
receiving, from the device, additional event data, each event in the additional event data having a timestamp that corresponds to a first time at least a threshold amount of time prior to the second time, the delta between the first time and the second time having occurred at least in part based on the device having been incapable of establishing the connection;
determining whether the additional event data is valid;
responsive to determining that the additional event data is valid, merging the additional event data into the existing event data at the event database; and
updating the medical decision based on the merged event data.

18. The computer system of claim 17, the actions further comprising:
receiving an audit indication for the medical decision;
retrieving a decision timestamp associated with the medical decision;
retrieving, from the electronic patient record, decision event data corresponding to a merge timestamp, the merge timestamp indicating when the decision event data was available for determining medical decisions; and
sending the decision event data for display on the medical audit device.

19. The computer system of claim 17, wherein the existing event data corresponds to a field in the electronic patient record and merging the additional event data into the existing event data based on the timestamp comprises:
receiving a corresponding timestamp of corresponding event data that includes event data that refers to the field;
comparing the timestamp of the additional event data to the corresponding timestamp of the corresponding event data that includes event data that refers to the field; and
responsive to determining that the timestamp is later than the corresponding timestamp, replacing the corresponding event data with the additional event data in the field of the electronic patient record.

20. The computer system of claim 17, wherein receiving, from the device previously incapable of providing event data, additional event data comprises:
receiving, from the device, one or more events in the additional event data, each event having a timestamp manually set at the device; and
synchronizing the timestamps with a reference clock of a device with which the medical decision was made.
